(19) United States
(12) Patent Application Publication
(10) Pub. No.: US 2015/0045688 A1
(43) Pub. Date: Feb. 12, 2015

(54) NARDI NEUROTYPE PROFILER SYSTEM

(71) Applicant: Dario Nardi, West Hollywood, CA (US)

(72) Inventor: Dario Nardi, West Hollywood, CA (US)

(73) Assignee: Dario Nardi, West Hollywood, CA (US)

(21) Appl. No.: 13/964,051

(22) Filed: Aug. 10, 2013

(51) Int. Cl. A61B 5/16; A61B 5/00; A61B 5/0476

(52) U.S. Cl. CPC A61B 5/167; A61B 5/0476; A61B 5/6803; A61B 5/742
USPC 600/544

(57) ABSTRACT

This Invention is a direct neurological assessment that generates scores for psychological types and neurological profiles. ASSESSMENT: Brain-mapping data is generated by EEG testing on multiple human subjects while performing tasks related to specific brain regions. Scores are calculated, algorithms developed, and visual diagrams drawn. The data is compared to known functions for specific brain neocortex regions, and verified by independent testing for cognitive/personality types by statistical analysis. TYPING: This process determines brain patterns for specific neocortex regions, synchronous circuit patterns between regions, and hemispheric balance. Patterns are identified and grouped into eight psychological types. PROFILES: Results include: (1) scores as raw values or percentages; (2) list of brain-mapped pattern characteristics; (3) colored or shaded map of brain region activity; (4) diagram of synchronous brain circuit patterns; (5) ranking psychological types into dominant, secondary, and recessive; and (6) a descriptive neurological and psychological profile. Applications include: counseling, business and education.

### CROSS-REFERENCE TO RELATED APPLICATIONS

This application claims priority to the Provisional Patent Application No. 61/682,242, filed Aug. 10, 2012

### BACKGROUND OF THE INVENTION

1. Field of Invention

This invention describes a novel psychometric instrument, derived from brain mapping, that generates a neurological or cognitive type profile (one of eight types) for an individual.

2. Prior Art

There are multiple problems with previous psychological evaluation systems. Most psychometric instruments assume a normal (typical) population and are self-assessments rather than actual skills tests. Whereas most actual tests typically focus on reasoning and memory, but do not assess for psychological or cognitive type. Many instruments test for traits; but traits are not types. Finally, the problem with most psychological evaluation systems is that they are based on indirect evidence of cognitive or personality patterns—not on neuroscience. Some involve psychometric instruments (scorable questionnaires), sortable card systems, or computer programs. Other researchers (Alcock, Gram, and Laposky) have attempted correlations between EEG and psychological type. But each failed to find meaningful results, beyond minor isolated correlations, likely because they engaged in short experimental sessions and used the wrong parameters. Still others used brain mapping to evaluate a person's mental state when responding to a traditional psychometric instrument. However, none were able to find system-wide correlations for either psychological evaluation, psychological type, or skills assessment, or to develop any other definitive systems. Hence, no other inventor has successfully formulated a psychometric instrument to assess cognitive type based on brain mapping.

### Prior Patents

### Chronological Order

A patent search shows ten (10) psychological typing systems as patents or pending patent applications. These include:
Shovers, Aaron: “Personality Analyzer”, U.S. Pat. No. 5,696,981, Dec. 9, 1997.
Bryce, Nathan & Kesterson, Russell: “Personality Testing Apparatus and Method, U.S. Pat. No. 5,702,253, Dec. 30, 1997.
Newton, John & Barth, Willard: “Method to Manage Marketing and Sales Data of E-Commerce Clients”, Patent Application #2003/0149572 A1, Aug. 7, 2003.
Hewson, Roger, Raymond, M. E. “Developing The Twelve Cognitive Functions of Individuals”, Patent Application #2005/0181339 A1, Aug. 18, 2005.
Tan, Ah Thau: “Psychometric Assessment Tool and Method for Interpreting Human Personality and Human Behavior.” Patent Application #2007/0048706 A1, Mar. 1, 2007.
Tieger, Paul: “System and Method for Improving Communications With A Patient.” Patent Application #2007/0250349 A1, Oct. 25, 2007.
Chan, John Lap Man: “System and Device for Determining Personality Type,” U.S. Pat. No. 7,950,664 B2. May 31, 2011.
Jung, Leuthardt, Levien, Lord, Malamud, Rinaldo, and Wood: “Methods and Systems for Indicating Behavior in a Population Cohort”, Patent Application #2009/0164403 A1, Jun. 25, 2009.
Toole, Sara Elizabeth: “Personality Profile Markers for Targeted Ads as a Method and a System”, Patent Application #2011/0276408 A1, Nov. 10, 2011.
Bonnstetter, Bill: “Validating Self-Reporting With Brain Waves”, Patent Pending Ser. No. 13/468,490. Filed 2012.

### Prior Psychological Typing Systems

A search of the literature shows multiple psychological typing systems, as books, articles, or psychometric instruments (scorable questionnaires). Only one, the oldest prior system, is for cognitive types and is inspired by (but not directly based on) neuroscience. These include:
Four (4) Quadrant Cognitive Types: C. Victor Bunderson, Dissertation: The Validity of the Herrmann Brain Dominance Instrument, published by Herrmann International, 1985.
Four (4) DiSC Social Styles: Marston, William M. (1928). Emotions of Normal People. K. Paul, Trench, Trubner & Co. Ltd, 1928.
Four (4) temperaments, Berens, Linda. An Introduction to the 4 Temperaments—4.0 Telos Publications, 2010.
Five (5) Personality Factors: Costa, P. T., Jr. & McCrae, R. R. Revised NEO Personality Inventory (NEO-PI-R) and NEO Five-Factor Inventory (NEO-FFI) manual. Odessa, FLa.: Psychological Assessment Resources, 1992.
Five (5) Personality Factors: Costa, P. T., Jr. & McCrae, R. R. Revised NEO Personality Inventory (NEO-PI-R) and NEO Five-Factor Inventory (NEO-FFI) manual. Odessa, FLa.: Psychological Assessment Resources, 1992.
Eight (8) Jungian Mental Functions: Jung, Carl. Psychological Types. Reprint, Princeton, N.J.: Princeton University Press, 1960.
Nine (9) Enneagram Types: Riso, Don Richard and Hudson, Russ. Personality Types: Using the Enneagram for Self-Discovery. Mariner Books, 1996.
Myers & Briggs: (16 types) MBTI (Myers-Briggs Type Indicator Instrument®) Isabel Briggs Myers, Mary H. McCaulley, Naomi L. Quenk, Allen L. Hammer. MBTI Manual: A guide to the development and use of the Myers Briggs type indicator (3rd ed). Consulting Psychologists Press, 1998.

### Prior Related Brain Research

To create a comprehensive brain-based Invention for cognition or personality, a wide range of published research in books and articles were reviewed. Here is a sample.
Colin G DeYoung, Jacob B Hirsh, Matthew S Shane, Xenophon Papademetris, Nallakkandi Rajeevan, and Jeremy R Gray. “Testing Predictions From Personality Neuroscience: Brain Structure and the Big Five”. Psychological Science, 21(6) (pages 820-828), 2010.
D Erik Everhart, David W. Harrison. “Hostility Following Right CVA: Support for Right Orbital Frontal Deactivation and Right Temporal Activation”. ISNR.
Ginette C Blackhart, John P Kline “Individual differences in anterior EEG asymmetry between high and low defensive individuals during a rumination/distraction task”. Personality and Individual Differences, 39 (pages 427-437), 2005.
J M Kilner, J L Marchant, and C D Frith. “Modulation of the mirror system by social relevance”. Social Cognitive and Affective Neuroscience, Vol. 1 (pages 143-148), 2006.
Marvin Zuckerman. Psychobiology of Personality, 2nd edition. Cambridge University Press, April 2005.
Norbert Jausovec. “Differences in Cognitive Processes Between Gifted, Intelligent, Creative and Average Individuals While Solving Complex Problems: An EEG Study”. Elsevier Science, 2000. (Available online: www.sciencedirect.com).
Peter C Gram, Bruce R Dunn, and Diana Ellis. “Relationship Between EEG and Psychological Type”. Journal of Psychological Type, Issue 5, November 2005.
R. W Thatchera, P. J Krausea, M Hrybyka. “Cortico-cortical associations and EEG coherence: A two-compartmental model”. Electroencephalography and Clinical Neurophysiology, Vol. 64, Issue 2, August 1986, pages 123-143.
Tetsuto Minami, K Goto, M Kitazaki, and S Nakauchi. “Asymmetry of P3 amplitude during oddball tasks reflects the unnaturalness of visual stimuli”. NeuroReport, Vol. 20 (pages 1471-1476), 2009.

### The Author's Prior Art (Chronological Order)

(1) Nardi, Dario: ISCA (Interstrength Cognitive Assessment′) is a psychometric instrument. The white paper describing it was published as Nardi, Dario: The Interstrength Cognitive Assessment: Development of a validated cognitive development psychometric (Research report 2), online in 2006. The ISCA is the psychometric assessment that Nardi developed to profile the eight mental types as defined by Carl Jung (1875-1961). Jung described mental functions (cognitive processes) based on his qualitative observations and conjecture. The ISCA reports percentage uses of specific cognitive processes, according to Jung's theories, based on Nardi's questionnaire. The ISCA does NOT include: (1) methods based on brain-mapping or MBTI; or (2) results describing detailed type profiles; or (3) results describing neurological skills profiles. The ISCA does NOT include Nardi's subsequent original research, and is not included in his provisional patent application or either non-provisional patent application related thereto.
(2) A related but separate Invention, the Neurological Performance Quotient™ (or Neuro-PQ), was first published online on Jan. 19, 2012. It was incorporated into Dario Nardi's Provisional Patent Application No. 61/682,242 titled Nardi Neurotype System & Neurological Performance Quotient, filed Aug. 10, 2012. It is being submitted as a separate Patent Application from the Nardi NeuroType System. This invention is a psychometric instrument, based on brain mapping and its known functions, that generates an assessment of a person's neurological skills. The NeuroPQ is objective and substantially different in subject matter from Nardi's other Inventions, (1) the ISCA and (3) the Nardi NeuroType Profile System.
(3) The current Invention, Nardi's NeuroType Profile System (or NeuroType Profiler), incorporated herein, is a psychological typing system by author Dario Nardi, consisting of eight types. It was first published in his related book: “Neuroscience of Personality,” Radiance House, Los Angeles, copyright July 2011, released Aug. 12, 2011. It was released and introduced to professionals at the APTi Biennial Conference (Association of Psychological Type International) on Aug. 12, 2011, on the same day that he was the keynote speaker there. It also was incorporated into Dario Nardi's Provisional Patent Application No. 61/682,242 titled Nardi Neurotype System & Neurological Performance Quotient, filed Aug. 10, 2012. It is herein submitted as a separate Patent Application from the Neuro-PQ. This Invention, the NeuroType Profiler, is a psychometric instrument, an assessment process using EEG brain-mapping, with verification from the scientific literature and a psychometric instrument, such as the ICSA (Nardi) or the MBTI personality type instrument, to assess a person's dominant cognitive type (one of eight). It reports brain-mapping details in percentages, as dominant brain-mapping traits, and as detailed textual profiles, consisting of cognitive and personality and physical traits. While this shares many brain-mapping methods with the Neurological Performance Quotient, the topics and results are substantially different. The Neuro-Type Profiler is objective and substantially different in methods of development, presentation of results, and psychological profiles from the ISCA.

### SUMMARY OF THE INVENTION

This Invention, Nardi NeuroType Profiler System™ (or NeuroType Profiler), is a novel psychometric process that generates scores for a cognitive typing system. The Invention is based on brain mapping, the known functions of mapped regions in the brain neocortex, and independent verification by cognitive-personality type testing. This direct neurological assessment for type, and any indirect assessments derived from it, constitute a new invention. METHODS: The NeuroType profile may be generated directly from brain mapping and novel algorithms, or from indirect assessments derived from brain-mapping. Preferably the brain-mapping data is generated by an EEG machine, data analyzed, algorithms developed, and visual diagrams and numeric scores developed to convey patterns. Optionally, derivative assessments could include a psychometric instrument (questionnaire with report), or other instrument, generated from previous brain-mapping data, patterns, and functions. RESULTS: Preferably brain mapping assessment is administered by a professional to determine cognitive types, synchronous circuit patterns, and hemispheric balance. Results preferrably include: (1) a list of brain-mapped pattern characteristics; (2) a colored or shaded map of brain region activity; (3) diagrams of synchronous brain circuit patterns; (4) scores as raw values or percentages; (5) ranking cognitive types into dominant, secondary, and recessive; and (6) descriptive profiles for relevant factors. A derivative assessment would be self-administered to assess cognitive type, and results could include: scores, ranking of cognitive types, and descriptive profiles. CONCLUSIONS: The Invention is a useful, novel, and unobvious process that accurately assesses neurological and cognitive type. Commercial applications include: clinical counseling, business, and education.

### DRAWINGS

FIG. 1, “Nardi-brain-map.pdf”, is an illustration of a birdseye view of the brain, portioned into 16 to 20 assessable sections, showing neocortex activity on a human individual. In the Invention this map is used for both methodology and reporting results.

FIG. 2, “Nardi-circuit-diagram.pdf”, illustrates pair-wise linkages in the neocortex derived from brain mapping for the purpose of assessing cognitive type.

### OBJECTS & ADVANTAGES

The object of the invention is to provide a psychometric instrument for assessing an individual's neurological-cognitive type. The advantages include:

1.) The Invention is more accurate because it employs objective brain mapping as its basis.
2.) The functions of each brain region are based on published scientific research.
3.) The Invention is verified by highly vetted psychometric instruments (ISCA or MBTI).
4.) The Invention measures and ranks sixteen to twenty areas of brain cognitive activity.
5.) The Invention determines longterm synchronous networks in the brain neocortex.
6.) The Invention determines hemispheric balance of the brain.
7.) The Invention determines an individual's dominant and secondary cognitive types.
8.) The Invention determines an individual's recessive cognitive types.
9.) The Report provides scores, lists, maps, circuit diagrams, and written profiles.

### DETAILED DESCRIPTION OF THE INVENTION

### Preferred Embodiments

The most preferred embodiments comprise:

### 1. Neurotype Profiler System™

A method of formulating a neurologically-based cognitive typing system, derived from brain mapping, for the purpose of determining an individual's neurological-cognitive type, comprising the following steps:
(a) Conducting brain wave mapping on human subjects to identify the levels of intensity of specific brain wave patterns in and across regions with known functions, while administering tasks related to specific brain regions;
(b) Researching and developing a table of neurological functions for specific regions of the brain neocortex, and linking said functions to the brain mapping data from step (a).
(c) Assessing psychological traits/types and/or cognitive skill levels of each subject;
(d) Verifying the brain mapping data from step (a) and related functions (step b) with a psychometric assessment from step (c), using appropriate statistical methods to develop and/or verify data;
(e) Identifying patterns in the data (step d) to group data into eight different cognitive types, to develop a cognitive typing system, for the purpose of determining the cognitive type of a future individual;
(f) Identifying supplementary patterns in the data (step d) for synchronous activation (coherence events) to refine the cognitive types;
(g) Identifying supplementary patterns in the data (step d) for hemispheric balance to refine the cognitive types;
(h) Developing a comprehensive report wherein results can be expressed in multiple formats and given to individuals to identify their neurological and cognitive patterns.

### 2. Brain-Mapping Method

In the preferred method brain wave mapping is conducted by electro-encephalographic (EEG) brain wave mapping on human subjects, or a comparable device, wearing a wired cap connected to a stationary monitor, for measuring and recording the electrical intensity of specific neocortical regions, each having a designated code and known function, and identifying specific brain wave patterns, which manifest in or across specific neocortical regions, in response to a variety of questions and stimuli administered by a researcher or psychotherapist, over a two to three hour period. The minimum number of subjects preferred is 40; whereas 80 subjects is still more preferred,

### 3. Brain-Mapping Alternate Method

In an alternate method brain wave mapping is conducted on a subject who is fitted with a wireless EEG headset that connects to a wearable or stationary monitor, for measuring and recording electrical intensity of specific neocortical regions, each having a designated code and known function, and wherein brain wave patterns, which manifest in or across specific neocortical regions, are generated in response to ordinary daily activity that is recorded in an activity log, and is periodically monitored by a researcher or psychotherapist.

### 4. Define Functions of Neocortex Regions

The preferred method of researching and developing a table of neurological functions or skills for specific regions (or set of regions) of the brain neocortex, and linking said functions with the brain wave mapping data; and wherein each region is designated by a code name, a skill set, and a brief profile defining psychological and neurological functions, as shown in Table 1.

TABLE 1
Twenty Neocortex Regions and Their known Functions *

| REGION | SKILL SET | PROFILE |
|---|---|---|
| Fp1 | Chief Judge | Decide between options. Filter out distractions to stay positive. |
| | (Fp1-ext.) | Focus on achieving a goal. Organize with confidence. |
| | Proof Polisher | Detect an error. Provide a reason. Correct something to be self- |
| | (Fp1-int.) | consistent. Limit range of facial expressions. |
| Fp2 | Authentic | Seek new ideas or stimulation. Facilitate a group in an emergent |
| | Enthusiast | way. Show facial expressions that honestly convey emotions. |
| | (Fp2-ext.) | |
| | Process | Track your step in a process. Perceive you are done |
| | Manager | brainstorming. Apply to yourself new or unpleasant ideas. |
| | (Fp2-int.) | |
| F7 | Imaginative | Infer based on context/analogy. Imagine a place or time. |
| | Mimic | Mirror other's behavior. Ask “what-if.” Mentally play out |
| | | situation. |
| F8 | Grounded | Recall exact details. Speak a word with emphasis. Identify |
| | Believer | beliefs. Rate how much you like or dislike. Ignore context. |
| F3 | Deductive | Make logical deductions. Backtrack reasoning to correct an |
| | Analyst | error. Follow a chain of reasoning. Devise action steps. |
| F4 | Witty | Sense how well something fits a category. Link two concepts |
| | Classifier | together. Interpret or compose metaphors, “Get” abstract joke. |
| T3 | Precise | Compose complex sentences. Form proper words, grammar, |
| | Speaker | and usage. Analyzing content of speech. Manage sexual |
| | | impulses. |
| T4 | Intuitive | Attend to tone of voice, when it resonates, seems phony, or has |
| | Listener | powerful affect. Recall melodies. Manage hostile impulses. |
| C3 | Factual | Remember facts. Retrieve information and sequences. Draw |
| | Storekeeper | skillful charts, tables, and diagrams. Move or feel right side. |
| C4 | Flowing | Retrieve memories of beautiful things or places. Draw skillful |
| | Artist | realistic free-hand drawings. Move or feel whole body. |
| T5 | Sensitive | Sensitive to and curious of other's opinions of you. Analyze |
| | Mediator | faces. Adjust your behavior to appease. Feel embarrassed. |
| T6 | Purposeful | Notice abstract spatial relationships. Assign symbolic |
| | Futurist | meanings. Envision/predict future events. Recognize faces. |
| P3 | Tactical Navigator | Integrate vision with action. Physical sense of self, boundaries, |
| | (P3-anterior) | and objects. Navigate precisely thru space. Detect shadowy |
| | | threats. |
| | Number Cruncher | Read quickly. Skillful arithmatic performance and rote memory. |
| | (P3-posterior) | Use finger or pointer to focus. Notice odd-ball objects. |
| P4 | Body Balancer | Notice inner body sensations (hunger, etc). Sympathize (e.g. |
| | (P4-anterior) | feel another's pain). Maintain spacial orientation. Dress and |
| | | groom. |
| | Strategic Gamer | Juggle multiple variables. Weigh pros and cons, risks vs |
| | (P4-posterior) | rewards. Link distantly related data. Identify and apply |
| | | leverage (influence). |
| O1 | Visual | Understand 3D visuals. Trust visual data as aid to think. See |
| | Engineer | precisely how to disassemble, rotate, and reassemble objects. |
| O2 | Abstract | Evaluate visual aesthetic design of colors, shapes, styles, |
| | Impressionist | themes, etc. Notice body language as means to detect people's |
| | | character. |

* References: See Prior Art: Related Brain Research.

### 5. Psychometric Assessment

A psychometric instrument (scorable questionnaire) may be selected from the group consisting of: (a) the ISCA (Interstrength Cognitive Assessment™ by Dario Nardi), that determines eight cognitive types and profiles and/or Myers-Briggs 4-letter codes (16 types); (b) the MBTI (Myers-Briggs Type Indicator Instrument®) that determines sixteen personality types and profiles; (c) the MPTI (Majors Personality Type Indicator™) that determines sixteen personality types and profiles, and/or (d) any equivalent psychometric instrument.

### 6. Analyzing Data: Statistical Methods

Preferred statistical methods include the two commonly accepted methods for EEG research: (a) qualitative analysis, consisting of observations recorded in a lab book and/or on video, of recorded brain wave activity and behavior patterns; and (b) quantitative analysis, consisting of original algorithms and factor analysis based on recorded EEG session data. Factors for analysis include the anatomical brain region codes (16 or more codes). Factors for verification may include: relevant published scientific research, the eight cognitive function designations (ISCA, Nardi, most preferred), the sixteen personality designations (MBTI or MPTI), or any other equivalent psychometric instrument. Optionally, data may be analyzed to determine sub-regions of brain activity based on: (1) linkages between two or more brain regions; (2) regions that often activate simultaneously (coherence events); (3) a nullifying effect; and/or (4) an algorithm to evaluate the strength of sub-regions.

7. identifying Cognitive Types

Preferrably patterns in the data are identified by grouping the data into eight different types to develop a cognitive typing system. This consists of a set of eight psychological-cognitive types, each with corresponding designation, code, profile, associated personality types, and associated EEG patterns. These are designated: (1) Extraverted Sensing, (2) Introverted Sensing, (3) Extraverted Intuiting, (4) Introverted Intuiting, (5) Extraverted Thinking, (6) Introverted Thinking, (7) Extraverte Feeling, and (8) Introverted Feeling. These may be ranked using an algorithm, to identify a dominant type, secondary types, and recessive types.

### 8. Extraverted Sensing: Type 1

A method wherein type 1 is designated as “Extraverted Sensing” (Se), named as “Active Adapter” and defined as: Act quickly and smoothly to handle whatever comes up in the moment; it has a psychological profile described as: Excited by motion, action, and nature, adept at physical multitasking with a video game-like mind primed for action, often in touch with body sensations, bored when sitting with a mental/rote task, strong memory for details in context, use their whole brain to handle crises, may be impatient to finish; this type is associated with Myers-Briggs types ESFP and ESTP; this type is also associated with the EEG pattern designated Se as follows:
Region Fp2 score greater than or equal to Fp1 score;
Alpha-1 band (8.0-14.0 Hz) predominates over all other bands except during conditions below;
Signing name with non-preferred hand evokes lower average of frequency and amplitude across all regions compared to signing name with preferred hand;
Delta-2 band (1.0-3.0 Hz) predominates for 30+ seconds across all regions during one of the following tasks: engage in role-play (live interpersonal simulation) or engage in ball-toss (throw and catch tennis balls back and forth);
And/or resting state (presented with no tasks or stimuli) demonstrates very low amplitude (less than 5 microvolts) with constantly changing frequency (0 to 50 Hz).

### 9. Introverted Sensing: Type 2

A method wherein type 2 is designated as “Introverted Sensing” (Si), named as “Cautious Protector” and defined as: Review and practice in order to specialize and meet group needs; it has a psychological profile described as: Constant practice helps their brain specialize; they improve when watching a role-model or example, often in touch with body sensations, easily track where they are in a task, use their whole brain to review the task, strong memory for kinship and raw details, may over-rely on authority for guidance; this type is associated with Myers-Briggs types ISTJ and ISFJ; this type is also associated with the EEG pattern designated Si as follows:
Region Fp2 score greater than or equal to Fp1 score;
Alpha-1 band (8.0-14.0 Hz) predominates over all other bands except during conditions below;
Signing name with non-preferred hand evokes lower average of frequency and amplitude across all regions compared to signing name with preferred hand;
Delta-2 band (1.0-3.0 Hz) predominates for 30+ seconds across all regions during the following task: review a past experience with eyes closed;
And/or excluding regions Fp1 and Fp2, highest scoring region is F7, F8, T5 or O1 among all regions.

### 10. Extraverted Intuiting: Type 3

A method wherein type 3 is designated as “Extroverted Intuiting” (Ne), named as “Excited Brainstormer” and defined as: Perceive and play with patterns of relationships across contexts; it has a psychological profile described as: Thinks analogically, notices and applies patterns of relationships across contexts, stimuli are springboards to generate analogies and ideas, easily guesses details, adept at role-play and minoring others, may find it hard to stay on task, brain activity tends to look chaotic with many highs and lows at once; this type is associated with Myers-Briggs types ENTP and ENFP; this type is also associated with an EEG pattern designated Ne as follows:
Region Fp2 score greater than or equal to Fp1 score;
Alpha-1 band (8.0-14.0 Hz) does NOT predominate over all other bands except when engaging in sensory activity (e.g. listening to music) with eyes closed;
Signing name with preferred hand evokes lower average of frequency and amplitude across all regions compared to signing name with non-preferred hand;
Excluding regions Fp1 and Fp2, highest scoring region is F7, P4, T4 or O2 among all regions.
And/or resting state (presented with no tasks) demonstrates moderate to high amplitude (greater than 10 microvolts) with constantly changing frequency (0 to 50 Hz).

### 11. Introverted Intuiting: Type 4

A method wherein type 4 is designated as “Introverted Intuiting” (Ni), named as “Keen Foreseer” and defined as: Draw upon the whole brain to realize an answer to a novel problem; it has a psychological profile described as: Enters a very brief trance to answer problems, focuses on what will happen, uses their whole brain to foresee the future, manages their own mental processes and are aware of where they are in an open-ended task, may use a physical action or symbol to focus the mind, may over-rely on the unconscious; this type is associated with Myers-Briggs types INTJ and INFJ; this type is also associated with the EEG pattern designated Ni as follows:
Region Fp2 score greater than or equal to Fp1 score;
Alpha-1 band (8.0-14.0 Hz) is the LEAST active of all bands except when engaging in sensory activity (e.g. listening to music) with eyes closed;
Signing name with preferred hand evokes lower average of frequency and amplitude across all regions compared to signing name with non-preferred hand;
Delta-2 band (1.0-3.0 Hz) predominates for 30+ seconds across all regions during the following task: envision a future situation; And/or delta-2 band (1.0-3.0 Hz) predominates for 3+ seconds across all regions when asked to respond with an answer to a question for which the subject is unfamiliar.

### 12. Extraverted Thinking: Type 5

A method wherein type 5 is designated as “Extroverted Thinking” (Te), named as “Timely Builder” and defined as: Manage resources efficiently to quickly decide based on the evidence; it has a psychological profile described as: Highly efficient use of brain resources, focuses on word content, recalls facts and figures easily, and sees and manipulates images and then decides, adept at giving decisive explanations, may display confidence even when wrong, tends to utilize other brain regions only when those regions are truly needed; this type is associated with Myers-Briggs types ESTJ and ENTJ; this type is also associated with the EEG pattern designated Te as follows:

Region Fp1 score greater than Fp2 score;

Delta-1 band (0.0-0.5 Hz) predominates over all other bands except during conditions below;

Excluding regions Fp1 and Fp2, highest scoring region is F7, F8, T3 or O1 among all regions;

Score for region O1 greater than score for region O2;

And/or average score for all left hemisphere regions is greater than average score for all right hemisphere regions.

### 13. Introverted Thinking: Type 6

A method wherein type 6 is designated as “Introverted Thinking” (Ti), named as “Skillful Sleuth” and defined as: Reason multiple ways to objectively and accurately analyze problems; it has a psychological profile described as: Adept at deductive reasoning, defining and categorizing, weighing odds and risks, and/or navigating spaces, can shut out senses to think, tends to back-track to clarify thoughts, separates body from mind when arguing or analyzing, may quickly stop listening, relies on interior regions of neocortex; this type is associated with Myers-Briggs types ISTP and INTP; this type is also associated with the EEG pattern designated Ti as follows:

Region Fp1 score greater than Fp2 score;

Theta band (3.5-7.5 Hz) predominates over all other bands except during conditions below.

Excluding regions Fp1 and Fp2, highest scoring region is F3, F4, P3 or P4 among all regions;

Score for region O1 greater than score for region O2;

And/or average score over regions F3, F4, P3, and P4 is greater than or equal to the average score in regions T3, T4, T5, and T6.

### 14. Extraverted Feeling: Type 7

A method wherein type 7 is designated as “Extroverted Feeling” (Fe), named as “Friendly Host” and defined as: Evaluates and communicates values to enhance social relationships; it has a psychological profile described as: Attends keenly to how others judge them, quickly adjusts behavior for social harmony, likes to use adjectives to convey values, they often hold back the true degree of their emotional response about morals/ethics and communicate it only in words or ideas, they may be overly coerced or embarrassed; this type is associated with Myers-Briggs types ESFJ and ENFJ; this type is also associated with the EEG pattern designated Fe as follows:

Region Fp1 score greater than Fp2 score;

Theta band (3.5-7.5 Hz) NEVER predominates over all other bands;

Excluding regions Fp1 and Fp2, highest scoring region is F8 or T5 among all regions;

Region Fp2 score is greater than scores for F3, P3, P4, O1, and O2.

And/or average score over regions F3, F4, P3, and P4 is less than the average score in regions F7, T3, T4, and T6.

### 15. Introverted Feeling: Type 8

A method wherein type 8 is designated as “Introverted Feeling” (Fi), named as “Quiet Crusader” and defined as: Listen with your whole self to locate and support what is important; it has a psychological profile described as: Evaluates personal importance along a spectrum from love/like to dislike/hate, attentive and curious for what is not said, focuses on word choice and voice tone, hard to embarrass, often does not utilize feedback, may strongly respond to specific high-value words, uses their whole brain to listen to others; this type is associated with Myers-Briggs types ISFP and INFP; this type is also associated with the EEG pattern designated Fi as follows:

Region Fp1 score greater than Fp2 score;

Delta-2 band (1.0-3.0 Hz) predominates for 30+ seconds across all regions during the following task: listens to one other person;

Delta-2 band (1.0-3.0 Hz) predominates for 3+ seconds across all regions during the the following task: listen to group or environment prior to taking an action;

Excluding regions Fp1 and Fp2, highest scoring region is T3, T4, T6 or F8 among all regions;

And/or average score over regions T3, T4, T5, and T6 is greater than or equal to the average score over regions F3, F4, P3, and P4.

### 16. Assessing Synchronous Activation

A method wherein supplementary patterns in the data are analyzed to locate synchronous activation events during brain mapping and identify patterns of bio-entrainment between brain regions (coherence events), which can be used to support and refine the attributes of the eight cognitive types to improve accuracy, which is particularly useful on older subjects.

### 17. Assessing Hemispheric Balance

A method wherein supplementary patterns in the data are analyzed to identify hemispheric balance and bias (right brain vs left brain) during brain mapping, which can be used to support and refine the attributes of the eight cognitive types to improve accuracy, which is particularly useful for complex subjects.

### 18. Developing A Report

A method of developing a comprehensive report, wherein results can be expressed in multiple formats as: (1) a list of brain-mapped pattern characteristics; (2) a colored or shaded map of brain region activity; (3) diagrams of synchronous brain circuit patterns; (4) scores as raw values or percentages derived from algorithms; (5) ranking cognitive types into dominant, secondary, and recessive types; and (6) descriptive profiles of important attributes of each type.

### 19. Optional Psychometric Instrument

An optional method wherein the Invention process may be used to formulate a psychometric instrument (scorable questionnaire), or other derived instruments, to assess a future individual for neurologic or cognitive type and develop a related profile, without using brain mapping on the individual.

### 20. Clinical Applications

The Invention may be used for clinical applications, including evaluation and therapy, based on a person's psychological type/traits, cognitive skill levels, and associated psychological profile for a selected individual or group of individuals; wherein clinical evaluation of profiles may include styles of: attention, behavior, cognition, emotion, motivation, personality, and spirituality; and wherein clinical therapies may include: skills counseling, behavioral or emotional counseling, career counseling, cognitive development, couple's communication, family counseling, improvement of small group dynamics, and mind-body integration.

### 21. Business Applications

The Invention may be used for business applications, based on a person's psychological type/traits, cognitive skill levels, and associated psychological profile for a selected individual or group of individuals; wherein these may include: advertising and marketing, communication skills and team dynamics, consumer behavior, dating service compatibility, human-computer interaction, job placement, leadership and management, organizational development, political messaging, sales, skills development, social networking behavior, as well as media design for books, electronic pads or computer applications, film and television, magazines, questionnaires, and smart phones.

### 22. Educational Applications

The Invention may be used for educational applications, based on a person's psychological type/traits, cognitive skill levels, and any associated psychological profile, for a selected individual or group of individuals; wherein these may include: academic counseling, career counseling, media design for textbooks and electronic pad or computer applications, types of learners and learning modes such as sensory modalities (auditory, tactile, or visual), types of instructors and instructional methods and materials, academic strengths and weaknesses such as concrete verses abstract math learners.

### DETAILED DESCRIPTION OF THE INVENTION

### Examples of Methods

### Example 1

### Gather Demographic Data

Administer consent forms and demographic forms for subjects to complete. Ideal subject is right-handed and free of brain damage, takes no mind-altering drugs, suffers no current mental impairments due to recent drug or alcohol use, sleeplessness, or mental illness, and has a history that is free of drug and alcohol abuse.

### Example 2

### Administer Personality Assessments

Administer a personality-profiling tool and/or cognitive skills test such as Nardi's ISCA or the Myers-Briggs Type Indicator. Store results for later scoring and analysis.

### Example 3

### Administer EEG Session

Preparation:

Follow standard protocol for EEG preparation, calibration, and use. This study used a Mindset ms-1000 10/20 EEG machine with ECI brand Electro-Cap which records measurements for 16 (sixteen) neo-cortex regions. A similar 10/20 EEG model may also be used. Turn on automatic data recording. Ready lab notebook and related implements. The session should be held at a time of day that meets the subject's best energy level and held in a room with an outdoor view that is natural and non-distracting. During the session, administer a battery of tasks over a 2-3 hour period (ideally 45 minutes for each of 3 parts below, though up to 1 hour per part is okay if not desirable). After each task, debrief the subject by asking, “Please briefly describe your experience doing the task?” If that proves uninteresting, ask, “What did you see, hear, and/or feel during the task?” Ideally, the sum of tasks covers all cognitive skills associated with the neocortex.

Part A:

Administer pre-defined solo tasks. These tasks should, as a whole, demand analytic and holistic thinking, engage the senses in both analytic and holistic ways, and involve opportunities for decision-making, interpretation, and problem solving. Ideally, the tasks are progressive or comparative in nature. E.g., subject solves a series of ever-harder math problems, signs name with both preferred hand and non-preferred hand, or plays a game at slow and fast pace.

Part B:

Utilize confederates (lab assistants) to administer pre-defined social tasks. Tasks include games (e.g. card games), speed dating, group problem solving, creative construction (e.g. compose a story together given prompts), and role-play skits of familiar activities (e.g. e.g. confederate plays customer and subject plays employee). Include at least one task that requests reading someone else's emotions or intentions. When possible, entreat subject to stand during social exercises.

Part C:

Ask subject to engage in tasks most relevant to the subject's area of creative expertise (e.g. playing musical instrument for musician, drawing a picture for an illustrator, solving math proofs for a mathematician, etc.) This may require tools such as a guitar or keyboard. Ideally, subject performs the activity 4 ways: simple performance, rehearsed difficult performance, imagined performed (eyes closed and visual in the mind), and improvisational performance.

These are essential tasks in order to complete step 7 below.

Sign name with preferred and non-preferred hand.

Listen activity to someone else.

Listen to music with eyes closed.

Review past experience and future situation, with eyes closed.

Toss a ball back and forth with confederate.

Make a decision and explain the decision afterward.

Engage subject to merely sit and wait for 3 minutes.

Part D:

Record Notable Observations

As the subject performs the tasks in step 3, maintain a logbook of the subject's actions and utterances and also notable events on the EEG, with time codes, for later reference. This is a qualitative recording. Be sure to note subject's EEG during the essential tasks above.

### Example 4

### Calculate EEG Activity Scores

Review the EEG recording made during step 3 above. For each EEG region, calculate:

(a) Average amplitude for each region at each 1 minute interval.
(b) Average amplitude and related statistics (minimum, maximum, etc) over the whole session.
(c) Total representation of each frequency band (alpha, beta, delta, gamma, theta) for each region and for the entire neocortex over the whole session.
(d) How many minutes each frequency band dominated over the other bands.

These results can be used in various ways. Calculate the following:

(e) Over the whole recording, count the number of exceptional events in each region. A region's activity is notable when the dominant band is two or more bands higher than the average frequency band across the whole EEG at that time. Example: If region P3 is dominant beta (18.5-40 Hz) at time t and the average frequency of the rest of the neocortex is below 14.5 Hz (alpha 1 band or lower) at time t, then add +1 to the score for P3.
(f) Optionally, normalize the scores above to an average for comparison against other subjects. For example, adjust all scores such that the highest equals 100. Represent the scores as normalized percentages. The average of all scores equals 50.
(g) Optionally, calculate scores for sub-regions. For example, given a score for region P3, compare adjacent regions C3 and O1. If C3 is higher, the score for subregion P3-ant. equals the score for P3+ an off-set value while the score for subregion P3-pos. equals the score for P3− the same off-set value. Conversely, if O1 is higher, reverse the use of the off-set value.
(h) Utilize data from step 2 above to calculate scores for sub-regions. For example, regarding Fp1, consider whether the subject scored higher on extraversion or introversion in Example 2. If extroversion is higher, the score for subregion Fp1-ext. equals the score for Fp1+ an off-set value while the score for subregion Fp1-int. equals the score for Fp1− the same off-set value. Conversely, if the introversion is higher, reverse the use of the off-set value.

### Example 5

### Calculate EEG Synchronous Activation Events

Review the EEG recording made during step 3 above. For each region, calculate:

(a) Coherence events (synchronous activation): How often the region changed its frequency band with 1 or more other regions at the same time.
(c) A coherence score, which equals the sum of all amplitudes across all wavelengths for that region. Preferrably, normalize these scores for a fair comparison against other subjects. Represent the scores as normalized percentages, where the average of all scores equals 50. The lowest score should be equal to or greater than 0 and the highest score should be less than or equal to 100. A less preferred method is to not normalize scores.

These results can be used in various ways. Calculate the following:

(d) Number of coherence events that are left and right hemisphere only versus cross-hemispheres.
(e) Every possible pair of regions (Fp1-Fp2, Fp1-F7, Fp1-T3, etc), refer to their corresponding coherence events and generate a list from the most frequently occuring coherence event to the least frequently occuring coherence event.

Preferrably, normalize the number of coherence events to vary from 100 (most frequent pairing) to 0 (least frequent pairing). Also, partion the list into quartiles to learn the top quarter of events and bottom quarter of events. A less preferred method is to not normalize the number of coherence events and/or not partition the list, or partition it in a different manner.

As references, among 16 regions, there are 120 possible non-directional covariant pairings, where (16×15)/2=120, with 30 pairings in each quartile; and among 20 regions, there are 190 possible covariant non-directional pairings, where (20×19)/2=190, with approximately 47 pairings in each quartile. A non-directional pairing means that two regions change together without reference to which region might lead the change, and a covarient pairing means both regions change upward or both regions change downward, as opposed to varying inversely, when one region rises while another region falls.

Given more information, you may calculate coherent pairs in more detail such pairings where inverse change occurs.

(f) Note other coherent events including but not limited to: presence of inverse coherent pairings, frequency of events when all regions except Fp1 (the left prefrontal cortex) change together, and other notable occurances, such as when any three or more regions change together.
(g) Use the coherent pairings to calculate a score for each brain region (Fp1, Fp2, F7, etc), where the score draws from the total number of normalized events calculated in step (b) for a region.

Preferrably, normalize these scores for a fair comparison against other subjects. Represent the scores as normalized percentages, where the average of all scores equals 50. The lowest score should be equal to or greater than 0 and the highest score should be less than or equal to 100. A less preferred method is to not normalize scores.

(h) Perform any additional analysis desired.

### Example 6

### Determine & Report Cognitive Types

Use the results generated from Examples 4 and 5 above to determine scores for each cognitive type. When in doubt, compare scores generated from Examples 4(f) and 5(g) above, deferring to the higher scores when determining cognitive type. The profiles and criteria are summarized under Embodiments 8 through 15 above. Present the cognitive types in ranked order, from highest to lowest, with the highest as the subject's primary cognitive type, the second as a secondary type, and the bottom ranked two as recessive types.

### Example 7

### Prepare a Shaded or Colored Map

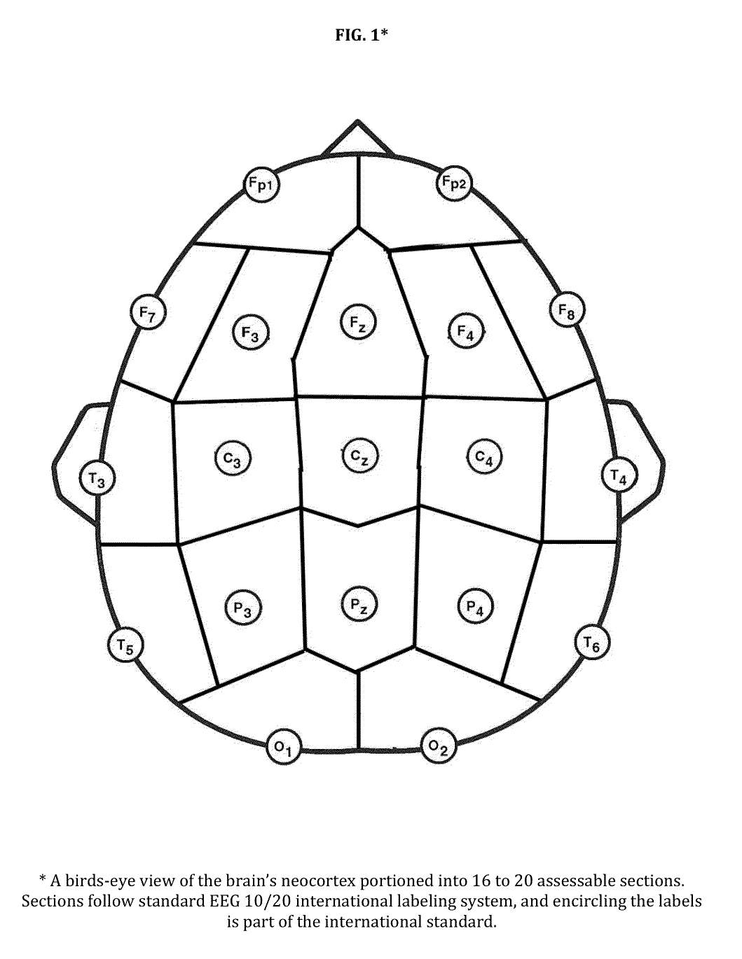

First, use the results in Example 4 above to prepare a colored or gray-scale illustration of the activity of the subject's neocortex. Start with a labeled bird's eye view of the neocortex as typically used during EEG research. See FIG. 1. Second, use the results in Example 5 above to prepare a colored or gray-scale illustration of the inferred activity of the subject's neocortex based on the circuit diagram, as described in Example 5, section (g). For both refer to the following legend below (Table 2). Compare the two maps. Note similarities and differences. Use a statistical test to calculate the degree of similarity.

TABLE 2
EEG Map Legend

| TIER | COLOR* | SHADE | SCORE |
|---|---|---|---|
| Very high | Red | White | 83% < x < 100% |
| High | Orange | Light gray | 66% < x < 84% |
| Medium-High | Yellow | Medium light gray | 49% < x > 67% |
| Medium-Low | Green | Medium dark gray | 33% < x < 50% |
| Low | Blue | Dark gray | 16% < x < 34% |
| Very Low | Black | Black | x < 17% |

*Use of color here indicates EEG amplitude (not frequency).

### Example 8

### Calculate & Report Hemispheric Usage

Calculate contribution of left hemisphere regions versus right hemisphere regions of the neocortex according to the scores gained in step 4 above.

Left hemisphere=(Fp1-ext.+Fp1-int.+F3+F7+C3+T3+T5+P3-ant.+P3-pos.+O1)/10

Right hemisphere=(Fp2-ext.+Fp2-int.+F4+F8+C4+T4+T6+P4-ant.+P4-pos.+O2)/10

### Example 9

### Prepare a Circuit Diagram

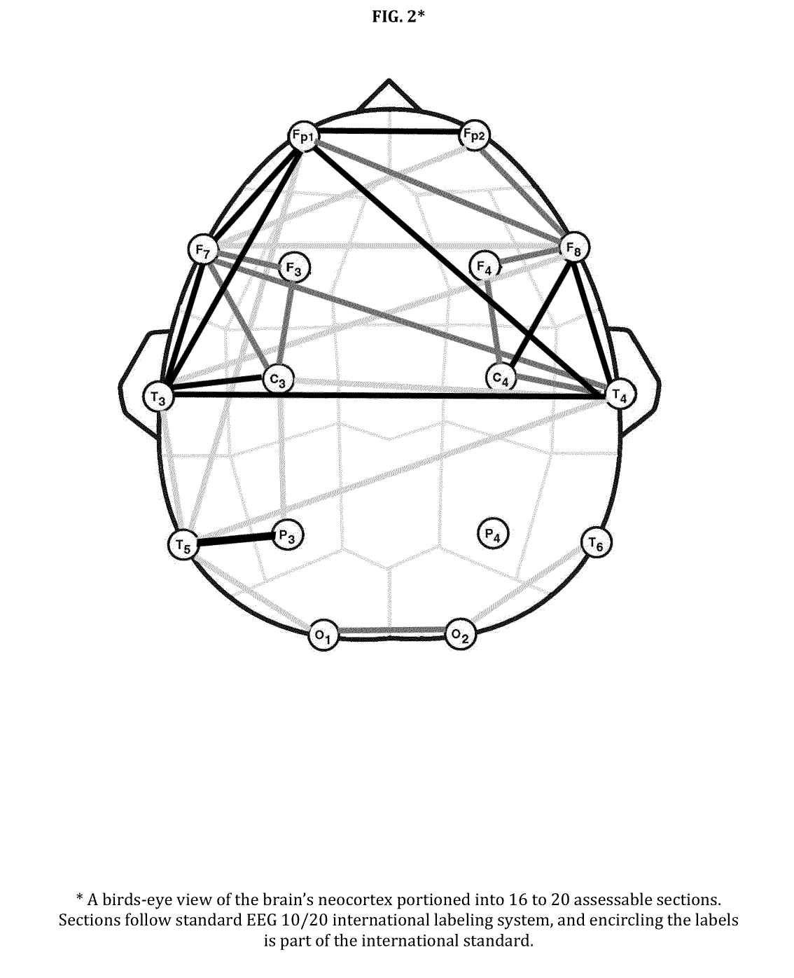

Use the results in step 5 above to prepare a diagram of the linkages between various regions of the subject's neocortex. Start with a labeled bird's eye view of the neocortex as typically used during EEG research. See FIG. 2. Refer to the following legend:

TABLE 3
EEG Circuit Diagram Legend

| TIER | LINE | RANK OF PAIRING |
|---|---|---|
| Always used | Black | Top 10 |
| Usually used | Dark gray | Rank 11-20 |
| More used | Light gray | Rank 21-30 |
| Less used | White, or not shown | Rank 31+ |
| Inverse use | Dotted gray | Any inverse links |

### Example 10

### Calculate & Report Circuit Balance

Use the results from step 5 above to calculate the report the percentage of pairings that fall into each category below:

(a) Left hemisphere=pairings that occur between regions located in the left hemisphere only, including regions Fp1, F7, F3, C3, T3, T5, P3-ant., P3-pos., and O1.
(b) Right hemisphere=pairings that occur between regions located in the right hemisphere only, including regions Fp2, F8, F4, C4, T4, T6, P4-ant., P4-pos., and O2.
(c) Horizontal cross-hemipshere=pairings between regions that sit directly across from each other including Fp1-Fp2, F7-F8, F3-F4, C3-C4, T3-T4, T5-T6, P3-ant.-P4-ant., P3-pos.-P4-pos., and O1-O2.
(d) Diagonal cross-hemisphere=non-horizontal pairings that occur between regions that are in different hemispheres such as Fp1-F8, Fp1-T4, Fp1-T6, and so forth.

Also report whether and how often all regions except Fp1 (the left prefrontal cortex) changed together, as described in part (f) of step 5.

### Example 11

### Report Other Notable Results

Refer to steps 4 and 5 above to report other calculated results, such as from part (a) of step 4, which is the average amplitude of all regions at 1 minute intervals, with which you may note when the subject tried various tasks performed in step 3 above to learn which tasks were more or less engaging for the subject.

### Example 12

### Infer Likely Alternate Psychometric Result

One can infer the results of another psychometric instrument such as MBTI 4-letter type code so long as it was used as part of validating the NeuroTypes Profiler. Refer to the subject's results from steps 4 through 11 above and match them against a database of psychometric results or a list of rules to locate best matches.

For example, infer a person's Myers-Briggs type code by comparing the person's scores, and results derived from the analysis of their scores, to a database of NeuroTypes Profiler results of persons of confirmed personality type. Display the percent match with each entry in the database as well as the best matches (or two or three best matches) that are likely type codes. Alternatively, to locate a Myers-Briggs type code, you can refer to a list of if-then rules and/or other criteria that determine the subject's best fitting type codes.

### Example 13

### Compare to Other Results

Compare the subject's results gained in steps 7 through 11 against the results of other persons, agregates of persons, or hypothetical agregates desirable for a career, group, job, task, or other context for an individual, group, or organizational report. If the subject has multiple NeuroTypes Profiler results over time, compare earlier and later results. You may visualize comparisons side-by-side or on a matrix or by any other means. You can use the results with other results to calculate statistics such as an average score.

### Example 14

### Suggest Recommendations

Determine the subject's primary context/s of need, whether business, clinical, or education. Refer to steps 7 through 12 above to locate psychological strengths and limitations. Client should utilize strengths and may wish to address limitations.

### DETAILED DESCRIPTION OF THE INVENTION

### Examples of Results

### Case History 1

### Example 1

### Demographic Data

Name: “Joe Smith” (derived from actual subject)

### Handedness: Right

### Sex: Male

### Age: 22

### Favored Testing Time of Day: Morning

### Example 2

### Personality Assessment Results

### Indicator Type Result: INFP

### Self-Selected Type Result: INFP

Cognitive Link: INFP=Primary Introverted Feeling with secondary Extraverted Intuiting.

### Example 3

### Administer EEG Session

Duration: 164 minutes
Tasks: Standard battery plus play musical instrument.
Data: Refer to audio recording, EEG data file, and written lab notebook.

### Example 4

### Notable Observations

Delta-2 band predominates for 30+ seconds across all regions when listening to one other person.
Alpha-1 band (8.0-14.0 Hz) was LEAST predominant band except when engaging in sensory activity (e.g. listening to music) with eyes closed.
Signing name with preferred hand evokes lower average frequency and amplitude across all regions compared to signing name with non-preferred hand.

### Example 5

### EEG Scores

TABLE 4
Subject 1's EEG Raw and Normalized Scores *

| | EEG | | | EEG | |
|---|---|---|---|---|---|
| REGION | SCORE** | SCORE | REGION | SCORE** | SCORE |
| Fp1-ext. | 290 | 27% | Fp2-ext. | 221 | 2% |
| Fp1-int. | 350 | 48% | Fp2-int. | 281 | 24% |
| F7 | 363 | 52% | F8 | 405 | 61% |
| F3 | 324 | 39% | F4 | 367 | 53% |
| T3 | 490 | 79% | T4 | 587 | 100% |
| T5 | 324 | 39% | T6 | 338 | 44% |
| C3 | 396 | 60% | C4 | 398 | 59% |
| P3-ant. | 375 | 54% | P4-ant. | 349 | 48% |
| P3-pos. | 215 | 0% | P4-pos. | 289 | 26% |
| O1 | 359 | 51% | O2 | 389 | 57% |

* Percentages may not average exactly 50% due to mathematical rounding.
**EEG scores for subregions of Fp1, Fp2, P3, and P4 are calculated using an algorithm.

### Example 6

### Make A Shaded/Colored Map

Make a colored or shaded map of the neocortex area based on EEG recordings. Subject l's neocortex map will look like the drawing of FIG. 1, but be colored or shaded to reflect the subject's individual EEG patterns. See color and shading Map Legend in Table 5 below.

TABLE 5
Subject 1's EEG Map Legend

| | | | | SUBJECT'S |
|---|---|---|---|---|
| TIER | COLOR | SHADE | SCORE | REGIONS |
| Very high | Red | White | 83% < x < 100% | T4 |
| High | Orange | Light gray | 66% < x < 84% | T3 |
| Med-High | Yellow | Med light | 49% < x > 67% | F7, C3, P3-ant., |
| | | gray | | O1, F8, F4, C4, |
| | | | | O2 |
| Med-Low | Green | Med dark | 33% < x < 50% | Fp1-int., F3, T5, |
| | | gray | | T6, P4-ant., |
| Low | Blue | Dark gray | 16% < x < 34% | Fp1-ext., Fp2- |
| | | | | int., P4-pos. |
| Very Low | Black | Black | x < 17% | P3-pos., Fp2-ext. |

### Example 7

### Hemispheric Balance

TABLE 6
Subject 1's Hemispheric Usage (Right Brain Vs Left Brain)

| HEMISPHERIC | | |
|---|---|---|
| BALANCE | PQ* | STYLES |
| Left Hemisphere | 46% | Analytical, focused, context-specific style. |
| Right Hemisphere | 54% | Holistic, diffuse, a-contextual style. |

*Percentages are normalized and may not average exactly 50% due to mathematical rounding.

### Example 8

### Top-Ranked Pairs

TABLE 7
Subject 1's Most Sychronous Links (Top 30 of 120 Regions)

| PAIRING | SCORE |
|---|---|
| T3-T4 | 100 |
| Fp1-T3 | 65 |
| Fp1-T4 | 60 |
| F8-C4 | 52 |
| F7-T3 | 50 |
| Fp1-F7 | 49 |
| F8-T4 | 45 |
| T3-C3 | 43 |
| Fp1-Fp2 | 41 |
| T5-P3 | 36 |
| C4-T4 | 35 |
| O1-O2 | 34 |
| Fp1-F8 | 33 |
| F7-F3 | 31 |
| F3-C3 | 28 |
| F4-C4 | 28 |
| F4-F8 | 28 |
| F7-C3 | 28 |
| F7-T4 | 28 |
| Fp2-F8 | 27 |
| F8-T3 | 25 |
| T3-T5 | 25 |
| C3-P3 | 24 |
| Fp1-T5 | 23 |
| C3-T4 | 22 |
| T4-T5 | 22 |
| T5-O1 | 22 |
| Fp2-F7 | 21 |
| F7-F8 | 20 |
| T6-O2 | 20 |

### Example 9

### Circuit Diagram

Prepare circuit diagram (synchonus links) of the neocortex area based on ranked pairs in Example 8 above. Subject l's neocortex map will look like the drawing of FIG. 2.

### Example 10

### Circuit Balance

TABLE 8
Subject 1's Hemispheric Balance (Unilateral Vs Crossover Links)

| Hemispheric Balance | Score | Styles |
|---|---|---|
| Left-Only Links | 43% | Analytical, focused, context-specific style. |
| Left-Right | 29% | Creative, synergistic, global style. |
| Cross-over Links | | |
| Right-Only Links | 27% | Holistic, diffuse, a-contextual style. |

* Percentages are normalized and may not average exactly 50% due to mathematical rounding.

Special Coherence Event: The following regions Fp2, F7, F3, F4, F8, C3, C4, T5, P3, P4, T6, O1, and O2 are synchronously active 9% of the time.

### Example 11

### Recommendations

### (a) Strengths:

4 Most-Active Regions:

T4, T3, F7 and F8.

Cognitive Skills:

Attends to voice tone, intentions, and ethics; also, attends to spoken content, word choice, and usage; likely strong musical skill; also, strongly felt beliefs, modest sense of self and cultural appropriateness, and good recall of important details; finally, displays rich imagination, skill with analogies and inferences, and mirrors others to learn or experience empathy.

NeuroTypes:

Primary Introverted Feeling (aka “Quiet Crusader”).

### (b) Limitations:

4 Least-Active Regions:

P3-pos, P4-pos., Fp2-ext., and Fp2-int.

Cognitive Limits:

Weak at mathematical calculations, may be a slow reader, and unlikely to strick easily to take and task; likely to make mistakes in weighing pros and cons, risks and uncertainties, lacking a strategic approach to problem solving; not particularly sponaneous in persuit of novel stimuli and does radiate enthusism or try to encourage participation in others; finally, may avoid or get lost in managing tasks (beginning, middle or end?), and may avoid introspection and self-change in response to criticism, failure, or new data.

Recessive NeuroType/s:

Extraverted Sensing (aka “Active Adapter”) and Introverted Sensing (aka “Cautious Protector”).

### (c) Suggestions:

Business:

Subject is best suited to job positions that requite quiet passion, imagination, active listening, and maybe manual dexterity. Consider musician. Subject is least suited to jobs that involve mathematics, strategy, high risk, or visual acuity such as engineer or entrepeneur.

Counseling:

Subject has social/cultural skills and focuses on listening for ethics. Subject may hold to high standards for relationships. Subject may at times be unaware of a partner's nonverbal feedback and will avoid self-change in response to negative feedback.

Education:

Subject is an auditory learner. Subject is best suited to fine arts, humanities, and social sciences. May be a slow reader. Subject is least suited to business, engineering, and mathematics.

### Patentability

This invention meets the criteria for patentability, as it is a novel, unobvious and useful process. It employs a novel method of physically mapping brain waves, and using the known functions of brain regions, with verification from a psychometric instrument, to determine neurological and cognitive types for individuals. It is an unobvious invention to psychologists or social workers, who traditionally use questionnaires or observation, and have little or no training in electronic evaluation. It is useful to assist clients to determine their psychological or cognitive processes and has commercial value for counseling, business, and education. It provides multiple benefits, such as: (1) eight ranked cognitive types (Sensing, Intuiting, Thinking, and Feeling, each in introverted or extroverted attitude) with scores and profiles; (2) supplementary circuit diagrams of synchronous activation of linked brain regions conveying which brain regions work together; (3) and assessment of hemispheric balance.

The details of the final invention are a trade secret, and have not been known or used by others until presented by the Inventor herein and in the provisional application. No scientific, commercial, or popular papers have been published containing the final research. No commercial products or sales have been made containing the details as described herein until recently by the Inventor. A U.S. Provisional Patent Application No. 61/682,242, was filed on Aug. 10, 2012, and no patents have been applied for elsewhere. Presentations have been made since that time.

### CONCLUSIONS, RAMIFICATIONS and ADVANTAGES

In conclusion, the NEUROTYPE PROFILER SYSTEM™ Invention provides a novel method of assessing neurological-cognitive type, synchronous brain wave events, and hemispheric balance by brain wave mapping; a detailed profile with graphics is provided. The ramifications indicate that cognitive and/or personality assessment may now move in the direction of a more technical approach involving more rigorous scientific validation. The advantages are multiple and include:

(1) The Inventor is a scientist, engineer, and professor trained in cognition and typing.
(2) The Invention is more accurate because it employs objective brain mapping as its basis.
(3) The functions of each brain region are based on published scientific research.
(4) The Invention is verified by highly vetted psychometric instruments (ISCA or MBTI).
(5) The Invention measures and ranks sixteen to twenty areas of brain cognitive activity.
(6) The Invention determines longterm synchronous networks in the brain neocortex.
(7) The Invention determines hemispheric balance of the brain.
(8) The Invention determines an individual's dominant and secondary cognitive types.
(9) The Invention determines an individual's recessive cognitive types.
(10) The Report provides scores, lists, maps, circuit diagrams, and written profiles.

1. A method of formulating a neurologically-based cognitive typing system, derived from brain mapping, for the purpose of determining an individual's neurological-cognitive type, comprising the following steps:
(a) Conducting brain wave mapping on human subjects to identify the levels of intensity of specific brain wave patterns in and across regions with known functions, while administering tasks related to specific brain regions;
(b) Researching and developing a table of neurological functions for specific regions of the brain neocortex, and linking said functions with the brain mapping data from step (a).
(c) Assessing psychological traits/types and/or cognitive skill levels of each subject;
(d) Verifying the brain mapping data (step a) and related functions (step b) with a psychometric assessment from step (c), using appropriate statistical methods to develop and/or verify data;
(e) Identifying patterns in the data (step d) to group data into eight different cognitive types, to develop a cognitive typing system, for the purpose of determining the cognitive type of a future individual;
(f) Identifying supplementary patterns in the data (step d) for synchronous activation (coherence events) to refine the cognitive types;
(g) Identifying supplementary patterns in the data (step d) for hemispheric balance to refine the cognitive types;
(h) Developing a comprehensive report wherein results can be expressed in multiple formats and given to individuals to identify their neurological and cognitive patterns.

2. The method according to claim 1, wherein brain mapping is conducted by electro-encephalographic (EEG) brain wave mapping on human subjects, or a comparable device, wearing a wired cap connected to a stationary monitor, for measuring and recording the electrical intensity of specific neocortical regions, each having a designated code and known function, and identifying specific brain wave patterns, which manifest in or across specific neocortical regions, in response to a variety of questions and stimuli administered by a researcher or psychologist, over a two to three hour period; and wherein the number of subjects ranges from 40 to 80.

3. An alternate method according to claim 2, wherein brain wave mapping is conducted on a subject who is fitted with a wireless EEG headset that connects to a wearable or stationary monitor, for measuring and recording electrical intensity of specific neocortical regions, each having a designated code and known function, and wherein brain wave patterns, which manifest in or across specific neocortical regions, are generated in response to ordinary daily activity that is recorded in an activity log, and is periodically monitored by a researcher or psychologist.

4. A method according to claim 1, of researching and developing a table of neurological functions or skills for specific regions (or set of regions) of the brain neocortex, and linking said functions with the brain wave mapping data; and wherein each region is designated by a code name, a skill set, and a brief profile defining psychological and neurological functions, as shown in Table 1 of the Description, entitled “Twenty Neocortex Regions and Their known Functions.”

5. A method according to claim 1, wherein a psychometric instrument (scorable questionnaire) may be selected from the group consisting of: (a) the ISCA (Interstrength Cognitive Assessment™ by Dario Nardi), that determines eight cognitive types and profiles and/or Myers-Briggs 4-letter codes (16 types); (b) the MBTI (Myers-Briggs Type Indicator Instrument®) that determines sixteen personality types and profiles; (c) the MPTI (Majors Personality Type Indicator™) that determines sixteen personality types and profiles, and/or (d) any equivalent psychometric instrument.

6. A method according to claim 1, wherein statistical methods include the two commonly accepted methods for EEG research: (a) qualitative analysis, consisting of observations recorded in a lab book and/or on video, of recorded brain wave activity and behavior patterns; and (b) quantitative analysis, consisting of original algorithms and factor analysis based on recorded EEG session data; and wherein factors for analysis include the anatomical brain region codes (16 or more codes); and wherein factors for verification can include: relevant published scientific research, the eight cognitive function designations (ISCA, Nardi, most preferred), the sixteen personality designations (MBTI or MPTI), or any other equivalent psychometric instrument; and wherein optionally, data may be analyzed to determine sub-regions of brain activity based on: (1) linkages between two or more brain regions; (2) regions that often activate simultaneously (coherence events); (3) a nullifying effect; and/or (4) an algorithm to evaluate the strength of sub-regions.

7. A method according to claim 1, wherein patterns in the data are identified and grouped into eight different types to develop a cognitive typing system, which consists of a set of eight neurological-cognitive types, each with corresponding designation, code, profile, associated personality types, and associated EEG patterns; and wherein these are designated: (1) Extraverted Sensing, (2) Introverted Sensing, (3) Extraverted Intuiting, (4) Introverted Intuiting, (5) Extraverted Thinking, (6) Introverted Thinking, (7) Extraverte Feeling, and (8) Introverted Feeling; and wherein these may be ranked using an algorithm, to identify a dominant type, secondary type, and recessive types.

8. A method according to claim 7, wherein type 1 is designated as “Extraverted Sensing” (Se), and defined as: Act quickly and smoothly to handle whatever comes up in the moment; it has a psychological profile described as: Excited by motion, action, and nature, adept at physical multitasking with a video game-like mind primed for action, often in touch with body sensations, bored when sitting with a mental/rote task, strong memory for details in context, use their whole brain to handle crises, may be impatient to finish; this type is associated with Myers-Briggs types ESFP and ESTP; this type is also associated with the EEG pattern designated Se as follows:
Region Fp2 score greater than or equal to Fp1 score;
Alpha-1 band (8.0-14.0 Hz) predominates over all other bands except during conditions below;
Signing name with non-preferred hand evokes lower average of frequency and amplitude across all regions compared to signing name with preferred hand;
Delta-2 band (1.0-3.0 Hz) predominates for 30+ seconds across all regions during one of the following tasks: engage in role-play (live interpersonal simulation) or engage in ball-toss (throw and catch tennis balls back and forth);
And/or resting state (presented with no tasks or stimuli) demonstrates very low amplitude (less than 5 microvolts) with constantly changing frequency (0 to 50 Hz).

9. A method according to claim 7, wherein type 2 is designated as “Introverted Sensing” (Si), and defined as: Review and practice in order to specialize and meet group needs; it has a psychological profile described as: Constant practice helps their brain specialize; they improve when watching a role-model or example, often in touch with body sensations, easily track where they are in a task, use their whole brain to review the task, strong memory for kinship and raw details, may over-rely on authority for guidance; this type is associated with Myers-Briggs types ISTJ and ISFJ; this type is also associated with the EEG pattern designated Si as follows:
Region Fp2 score greater than or equal to Fp1 score;
Alpha-1 band (8.0-14.0 Hz) predominates over all other bands except during conditions below;
Signing name with non-preferred hand evokes lower average of frequency and amplitude across all regions compared to signing name with preferred hand;
Delta-2 band (1.0-3.0 Hz) predominates for 30+ seconds across all regions during the following task: review a past experience with eyes closed;
And/or excluding regions Fp1 and Fp2, highest scoring region is F7, F8, T5 or O1 among all regions.

10. A method according to claim 7, wherein type 3 is designated as “Extroverted Intuiting” (Ne), and defined as: Perceive and play with patterns of relationships across contexts; it has a psychological profile described as: Thinks analogically, notices and applies patterns of relationships across contexts, stimuli are springboards to generate analogies and ideas, easily guesses details, adept at role-play and mirroring others, may find it hard to stay on task, brain activity tends to look chaotic with many highs and lows at once; this type is associated with Myers-Briggs types ENTP and ENFP; this type is also associated with an EEG pattern designated Ne as follows:
Region Fp2 score greater than or equal to Fp1 score;
Alpha-1 band (8.0-14.0 Hz) does NOT predominate over all other bands except when engaging in sensory activity (e.g. listening to music) with eyes closed;
Signing name with preferred hand evokes lower average of frequency and amplitude across all regions compared to signing name with non-preferred hand;
Excluding regions Fp1 and Fp2, highest scoring region is F7, P4, T4 or O2 among all regions.
And/or resting state (presented with no tasks) demonstrates moderate to high amplitude (greater than 10 microvolts) with constantly changing frequency (0 to 50 Hz).

11. A method according to claim 7, wherein type 4 is designated as “Introverted Intuiting” (Ni), and defined as: Mraws upon the whole brain to realize an answer to a novel problem; it has a psychological profile described as: Enters a very brief trance to answer problems, focuses on what will happen, uses their whole brain to foresee the future, manages their own mental processes and are aware of where they are in an open-ended task, may use a physical action or symbol to focus the mind, may over-rely on the unconscious; this type is associated with Myers-Briggs types INTJ and INFJ; this type is also associated with the EEG pattern designated Ni as follows:
Region Fp2 score greater than or equal to Fp1 score;
Alpha-1 band (8.0-14.0 Hz) is the LEAST active of all bands except when engaging in sensory activity (e.g. listening to music) with eyes closed;
Signing name with preferred hand evokes lower average of frequency and amplitude across all regions compared to signing name with non-preferred hand;
Delta-2 band (1.0-3.0 Hz) predominates for 30+ seconds across all regions during the following task: envision a future situation;
And/or delta-2 band (1.0-3.0 Hz) predominates for 3+ seconds across all regions when asked to respond with an answer to a question for which the subject is unfamiliar.

12. A method according to claim 7, wherein type 5 is designated as “Extroverted Thinking” (Te), and defined as: Manage resources efficiently to quickly decide based on the evidence; it has a psychological profile described as: Highly efficient use of brain resources, focuses on word content, recalls facts and figures easily, and sees and manipulates images and then decides, adept at giving decisive explanations, may display confidence even when wrong, tends to utilize other brain regions only when those regions are truly needed; this type is associated with Myers-Briggs types ESTJ and ENTJ; this type is also associated with the EEG pattern designated Te as follows:
Region Fp1 score greater than Fp2 score;
Delta-1 band (0.0-0.5 Hz) predominates over all other bands except during conditions below;
Excluding regions Fp1 and Fp2, highest scoring region is F7, F8, T3 or O1 among all regions;
Score for region O1 greater than score for region O2;
And/or average score for all left hemisphere regions is greater than average score for all right hemisphere regions.

13. A method according to claim 7, wherein type 6 is designated as “Introverted Thinking” (Ti), and defined as: Reason multiple ways to objectively and accurately analyze problems; it has a psychological profile described as: Adept at deductive reasoning, defining and categorizing, weighing odds and risks, and/or navigating spaces, can shut out senses to think, tends to back-track to clarify thoughts, separates body from mind when arguing or analyzing, may quickly stop listening, relies on interior regions of neocortex; this type is associated with Myers-Briggs types ISTP and INTP; this type is also associated with the EEG pattern designated Ti as follows:
Region Fp1 score greater than Fp2 score;
Theta band (3.5-7.5 Hz) predominates over all other bands except during conditions below.
Excluding regions Fp1 and Fp2, highest scoring region is F3, F4, P3 or P4 among all regions;
Score for region O1 greater than score for region O2;
And/or average score over regions F3, F4, P3, and P4 is greater than or equal to the average score in regions T3, T4, T5, and T6.

14. A method according to claim 7, wherein type 7 is designated as “Extroverted Feeling” (Fe), and defined as: Evaluates and communicates values to enhance social relationships; it has a psychological profile described as: Attends keenly to how others judge them, quickly adjusts behavior for social harmony, likes to use adjectives to convey values, they often hold back the true degree of their emotional response about morals/ethics and communicate it only in words or ideas, they may be overly coerced or embarrassed; this type is associated with Myers-Briggs types ESFJ and ENFJ; this type is also associated with the EEG pattern designated Fe as follows:
Region Fp1 score greater than Fp2 score;
Theta band (3.5-7.5 Hz) NEVER predominates over all other bands;
Excluding regions Fp1 and Fp2, highest scoring region is F8 or T5 among all regions;
Region Fp2 score is greater than scores for F3, P3, P4, O1, and O2.
And/or average score over regions F3, F4, P3, and P4 is less than the average score in regions F7, T3, T4, and T6.

15. A method according to claim 7, wherein type 8 is designated as “Introverted Feeling” (Fi), and defined as: Listen with your whole self to locate and support what is important; it has a psychological profile described as: Evaluates personal importance along a spectrum from love/like to dislike/hate, attentive and curious for what is not said, focuses on word choice and voice tone, hard to embarrass, often does not utilize feedback, may strongly respond to specific high-value words, uses their whole brain to listen to others; this type is associated with Myers-Briggs types ISFP and INFP; this type is also associated with the EEG pattern designated Fi as follows:
Region Fp1 score greater than Fp2 score;
Delta-2 band (1.0-3.0 Hz) predominates for 30+ seconds across all regions during the following task: listens to one other person;
Delta-2 band (1.0-3.0 Hz) predominates for 3+ seconds across all regions during the the following task: listen to group or environment prior to taking an action;
Excluding regions Fp1 and Fp2, highest scoring region is T3, T4, T6 or F8 among all regions;
And/or average score over regions T3, T4, T5, and T6 is greater than or equal to the average score over regions F3, F4, P3, and P4.

16. A method according to claim 1, wherein supplementary patterns in the data are analyzed to locate synchronous activation events during brain mapping and identify patterns of bio-entrainment between brain regions (coherence events), which can be used to support and refine the attributes of the eight cognitive types to improve accuracy, which is particularly useful on older subjects.

17. A method according to claim 1, wherein supplementary patterns in the data are analyzed to identify hemispheric balance and bias (right brain vs left brain) during brain mapping, which can be used to support and refine the attributes of the eight cognitive types to improve accuracy, which is particularly useful for complex subjects.

18. A method according to claim 1, of developing a comprehensive report, wherein results can be expressed in multiple formats as: (1) a list of brain-mapped pattern characteristics; (2) a colored or shaded map of brain region activity; (3) diagrams of synchronous brain circuit patterns; (4) scores as raw values or percentages derived from algorithms; (5) ranking cognitive types into dominant, secondary, and recessive types; and (6) descriptive profiles of important attributes of each type.

19. An optional method according to claim 1, wherein the Invention process may be used to formulate a psychometric instrument (scorable questionnaire), or other derived instruments, to assess a future individual for neurologic or cognitive type and develop a related profile, without using brain mapping on the individual.

20. A method according to claim 1, wherein the cognitive typing system, or a psychometric instrument derived from the Invention, can be used in the fields of clinical counseling, business, education, or other endeavors, to be administered to future individuals in those fields to generate cognitive type profiles.
